(19) United States
(12) Patent Application Publication
(10) Pub. No.: US 2020/0130917 A1
(43) Pub. Date: Apr. 30, 2020

(54) Ultrasound Transmission Gel Packet Having Internal Heat Source and Method of Use

(71) Applicant: Robert Steins, Seattle, WA (US)

(72) Inventor: Robert Steins, Seattle, WA (US)

(21) Appl. No.: 16/725,803

(22) Filed: Dec. 23, 2019

Related U.S. Application Data
(63) Continuation-in-part of application No. 15/638,199, filed on Jun. 29, 2017, now Pat. No. 10,556,734.

(51) Int. Cl. B65D 81/18; B65D 75/28; B65D 75/58; B65D 81/32; A61B 8/00

(52) U.S. Cl. CPC B65D 81/18; B65D 75/28; A61B 8/4272; B65D 81/3266; B65D 75/5805

(57) ABSTRACT

An Ultrasound Transmission Gel Packet Having Internal Heat Source and Method of Use. The gel packet is sized for a single use, rather than for multiple applications. The gel packets are not pre-heated, but rather are quickly heatable on demand. The gel packets may incorporate a variety of optional internal heating methods, including chemical activation, mechanical agitation and electrical activation, among others. The gel packets are sealed and therefore have extended shelf lives, and further are disposable after use.

This application is a continuation of application Ser. No. 15/638,199, filed Jun. 29, 2017, now pending.

### BACKGROUND OF THE INVENTION

### 1. Field of the Invention

This invention relates generally to medical devices and procedures and, more specifically, to an Ultrasound Transmission Gel Packet Having Internal Heat Source and Method of Use.

### 2. Description of Related Art

The use of ultrasound technology for the purpose of diagnostic imaging is performed in virtually every hospital and most urgent care facilities in the world. Furthermore, ultrasound scanning is conducted in most of the treatment areas within each health care facility. Because sound does not travel well in air, physicians and technologists performing an ultrasound examination routinely apply a conductive gel to the subject area prior to placing the ultrasound probe against the patient's body. For comfort and other safety reasons (e.g. thermal stress to neonatal infants¹), it is a common practice for health care facilities to pre-warm the conductive gel prior to patient application. FIGS. 1 and 2 provide the background of how the gel is heated. ¹ [symbol] “Procedural Hazards of Neonatal Ultrasonography” J Clin Ultrasound. 1986 June; 14(5):361-6.

Single-use gel packages are often used for dispensing ultrasound gel. FIG. 1 is a flowchart depicting the conventional single-use gel packet use method 10. Once obtained 100, the single-use gel packets are typically placed into a dedicated gel-pack warmer 102. When gel is needed in order to perform an ultrasound scan on a patient, the heated gel packet is retrieved from the warmer, opened, and the heated gel is applied to the patient 104. The expended gel packet is then discarded 106.

In some facilities or environments, multi-use dispensers (e.g. bottles or other containers) of ultrasound gel are employed. FIG. 2 is a flowchart depicting the conventional multi-use gel container use method 12. The multi-use container is obtained 108 and placed into the warming device 110, where it/they reside until gel is needed. When desired, the bottle is removed from the heating device 112, and the heated gel is either applied directly to the patient 134 (and then the bottle returned 136), or first into a cup or cloth 114, and then to the patient 118 (usually after returning the bottle to the heating device 116). This process is repeated until the bottle/container is empty or expired 122. Expired bottles 124 are discarded 126. Empty bottles 128 are cleaned and/or disinfected 130, refilled 132, and returned to service.

The problem with these conventional use methods is the prevention of gel contamination. Both methods include a shared or community heating device, typically a water bath or oven, which are known breeding grounds for pathogens. This can easily result in patients contracting a nosocomial infection.² While a number of procedures have been developed to curtail this risk, gel bottle warmers continue to be a significant source of nosocomial infection. ² [symbol] See Appendix I

What is needed is a more sanitary heated gel package and method of use.

### SUMMARY OF THE INVENTION

In light of the aforementioned problems associated with the prior devices and methods, it is an object of the present invention to provide an Ultrasound Transmission Gel Packet Having Internal Heat Source and Method of Use. The gel packet should be sized for a single use, rather than for multiple applications. The gel packets ideally should not be pre-heated, but rather should be quickly heatable on demand. The gel packets should incorporate a variety of optional internal heating methods, including chemical activation, mechanical agitation and electrical activation, among others. The gel packets should be sealed and therefore have extended shelf lives, and further should be disposed of after use.

### BRIEF DESCRIPTION OF THE DRAWINGS

The objects and features of the present invention, which are believed to be novel, are set forth with particularity in the appended claims. The present invention, both as to its organization and manner of operation, together with further objects and advantages, may best be understood by reference to the following description, taken in connection with the accompanying drawings, of which:

FIG. 1 is a flowchart depicting the conventional single-use gel packet use method;

FIG. 2 is a flowchart depicting the conventional multi-use gel packet use method;

FIG. 3 is a flowchart depicting a preferred method of use of the single-use individually-heatable gel packets;

FIG. 4 is a functional perspective view of a single-use gel packet having an internal heat source;

FIG. 5 is a diagram showing optional heating options for the internal heat source of the gel packet of the present invention;

FIG. 6 is a perspective view of a preferred embodiment of a chemically-activated version of the gel packet of FIG. 4;

FIG. 7 is a front view of a preferred embodiment of a single-use gel packet having electrified casing heat source;

FIG. 8 is a partial cutaway end view of the packet of FIG. 7; and

FIG. 9 are front views of several alternate versions of the single-use gel packet having an internal heat source.

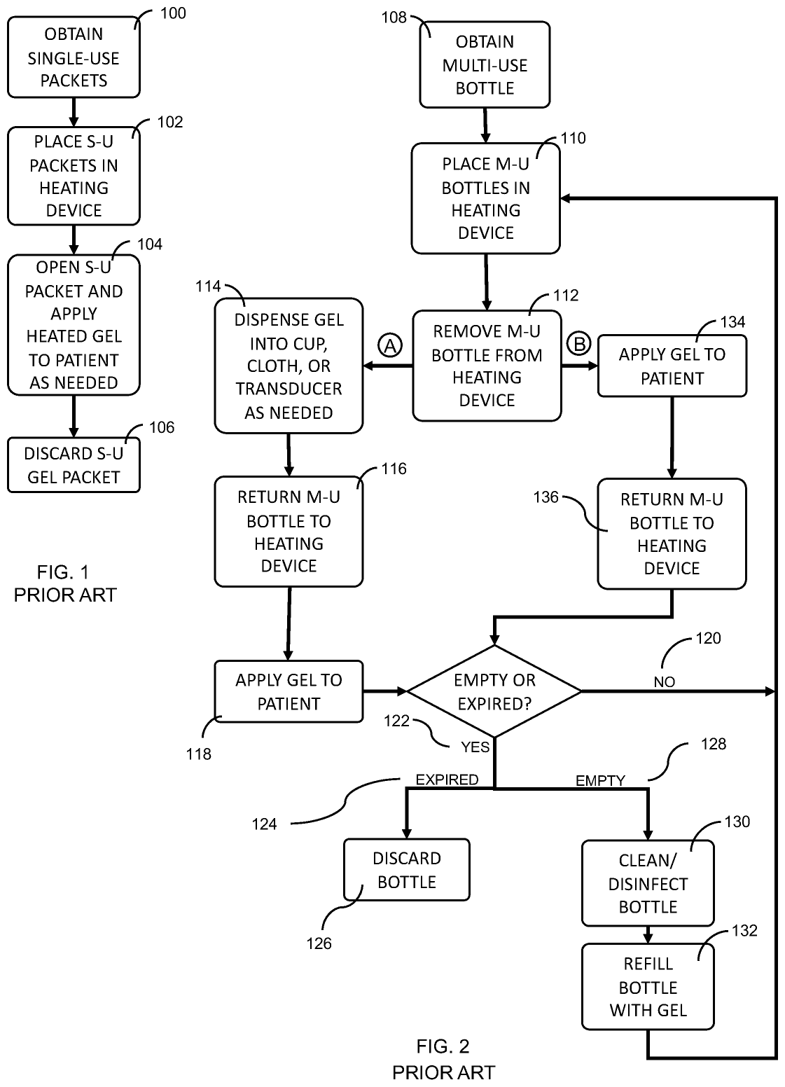

### DETAILED DESCRIPTION OF THE PREFERRED EMBODIMENTS

The following description is provided to enable any person skilled in the art to make and use the invention and sets forth the best modes contemplated by the inventor of carrying out his invention. Various modifications, however, will remain readily apparent to those skilled in the art, since the generic principles of the present invention have been defined herein specifically to provide an Ultrasound Transmission Gel Packet Having Internal Heat Source and Method of Use.

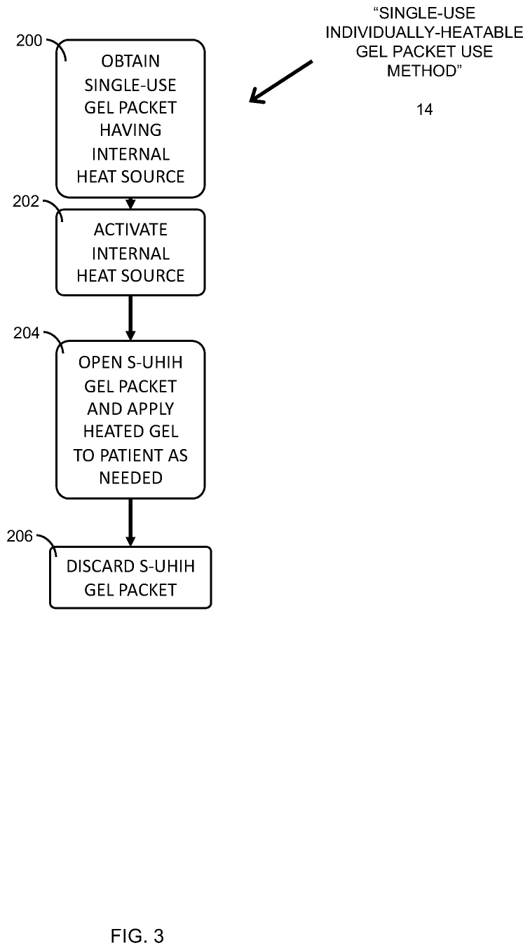

The present invention can best be understood by initial consideration of FIG. 3.³ FIG. 3 is a flowchart depicting a preferred method of use of the single-use individually-heatable gel packets 14. As discussed previously, the purpose of the instant method and device is to reduce the level of risk of nosocomial infection created by the process steps and the components involved in the heating of ultrasound gel. ³ [symbol] As used throughout this disclosure, element numbers enclosed in square brackets [ ] indicates that the referenced element is not shown in the instant drawing figure, but rather is displayed elsewhere in another drawing figure.

The initial step in this new process involves obtaining a single-use gel packet that has an internal heat source 200. The design specifics of the individually, internally-heated gel packet will be discussed below in connection with other drawing figures, however, it is sufficient to understand that there is no oven or other water bath involved in the packet-heating process. Each packet is individually heated on an as needed basis in order to minimize the growth and transfer of contaminating microorganisms, as has been shown to be a problem with the prior methods and devices. Consequently, the packages can remain in a hygienic environment until they are removed from the storage container for use.

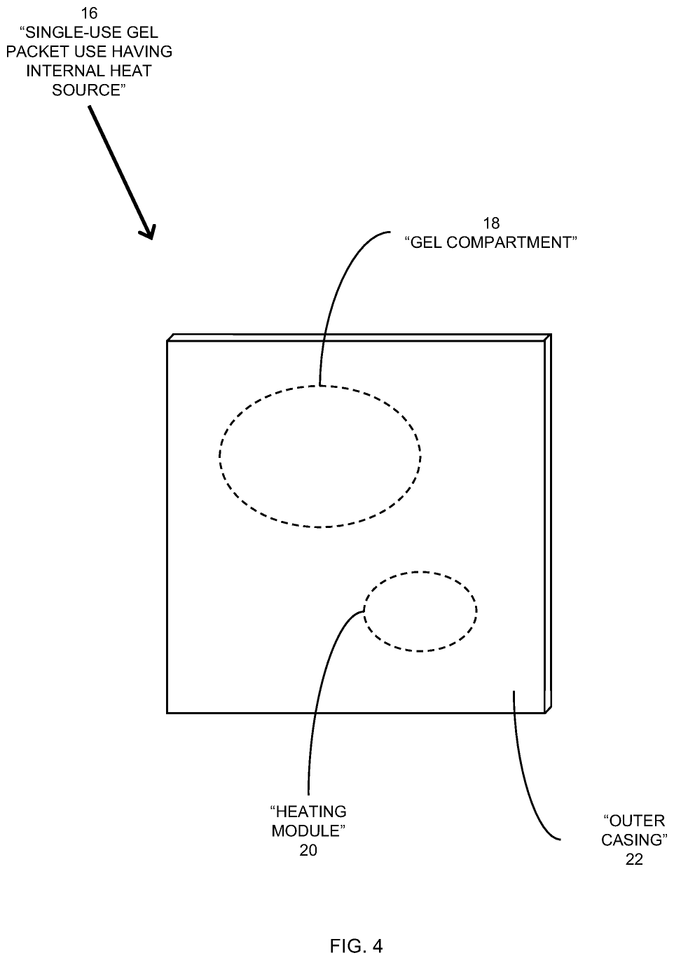

The user first obtains a single gel packet of the type described herein below 200. The internal heat source is then activated within the gel packet 202. Next, the operator (typically a sonographer or sonologist) opens the gel packet, dispenses the heated gel and applies it to the patient in the area to be scanned 204. Finally, the operator discards the empty gel package 206. The steps 200-206 are repeated for each dispensing of heated gel. It should also be noted that if the operator deems that heated gel is not desirable, he or she simply opens the packet and applies the gel without first heating the gel. This additional benefit means that the operator only requires a single source of gel packets, rather than being required to maintain separate inventories of heated and unheated gel packages. FIG. 4 introduces the gel packets used in this process 14.

FIG. 4 is a functional perspective view of a single-use gel packet having an internal heat source 16. The packet 16 is preferably has an outer casing 22 made from sterile, disposable materials. Within the casing, there is a gel compartment 18 and a heating module 20. As discussed below, the heating module 20 may actually be incorporated into the outer casing 22, such as to form the gel compartment 18, in certain versions.

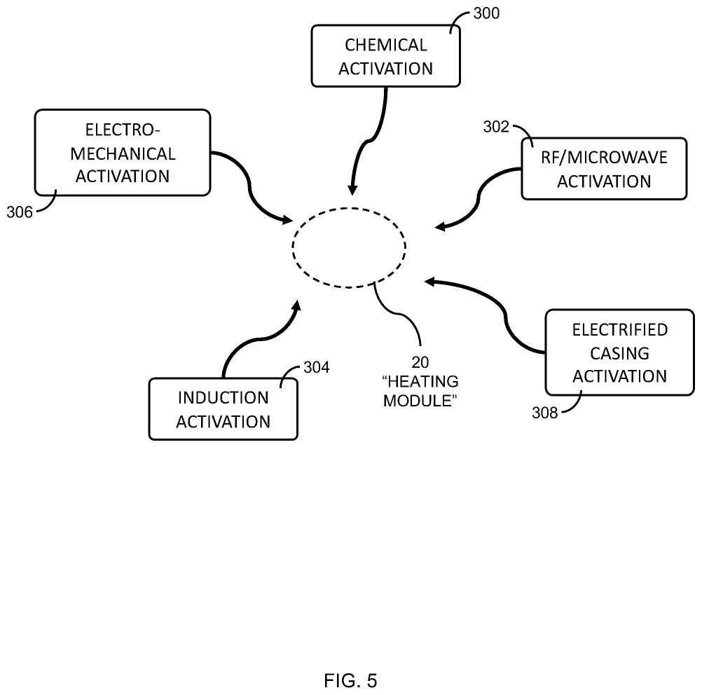

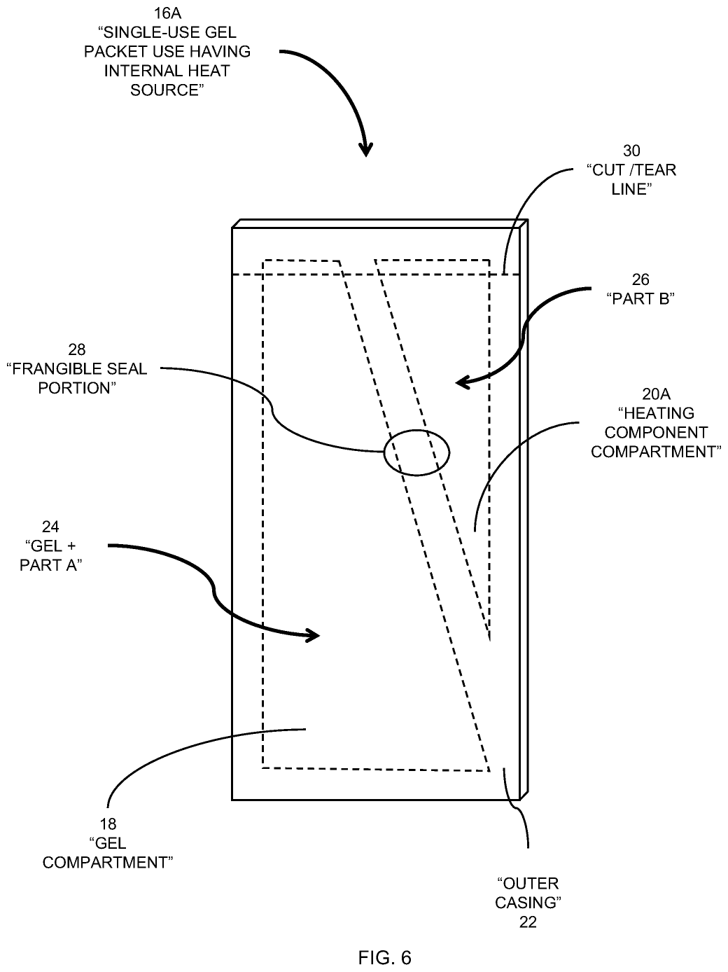

FIG. 5 is a diagram showing optional heating options for the internal heat source of the gel packet of the present invention. One preferred version of the heating module 20 is by chemical activation—that is to say that the gel in the compartment [18] would generally be mixed with a second chemical component (in a second gel compartment [18]). When mixed, the two components would generate a desirable amount of heat as a result of the exothermic reaction. This version is more fully described below in connection with FIG. 6.

The heating module 20 could also comprise electrical induction activation 304. This version may have coiled electrical filaments embedded within the casing [22] and/or gel compartment [18], or even as a series of filaments actually protruding into the gel compartment [18] itself. The heating mechanism could be via induction. If a gel package having such a heating module 304 is placed into a magnetic field, the field will cause current to flow through the electrical filaments. The filaments and package will be designed so that they will begin to heat up as current is generated, thereby heating the gel in the compartment [18] to the desired temperature. The magnetic field generator could be operated automatically to turn its magnetic field on when a new packet is placed in a predetermined location, and then turn off automatically either after a preset time is reached, or after a preset temperature is reached.

In another version, the heating module 20 could also comprise Joule or Ohmic heating 308. This version may produce heat when a resistive electrical conductor is activated by an electrical current. This may be realized by embedding resistive conductive layers within the multi-ply structure of the packet lining surrounded by insulative, thermally conductive plies. If the gel package with such heating module 308 is applied an electrical current to this embedded conductive layer, then the resistive conductive ply will generate Joule heating and transfer heat energy to the contained gel. The conductive layer and package would be designed so that the applied electrical current would occur when the package is placed in a predetermined location. The electrical current, and therefore Joule heating, would turn off automatically after a preset time or when desired gel temperature is reached so as to not present a shock or safety hazard to the user or patient.

In another version, the heating module 20 could heat through electro-mechanical means 306. In this embodiment, a piezoelectric device would be embedded within the casing [22] or would protrude into the gel compartment [18] itself. When connected to an electrical energy source, the piezoelectric device converts electrical energy into mechanical vibration, thereby generating heat in the gel.^{4,5} The energy source could be operated automatically to activate the piezoelectric element or elements when the packet is placed in a predetermined location, and then turn off automatically either after a preset time is reached, or after a preset temperature is reached. ⁴ [symbol] Eduardo Moros, ed., Physics of thermal therapy: fundamentals and clinical application, Imaging in medical diagnosis and therapy (Boca Raton, Fla.: CRC/Taylor & Francis, 2013), 81-82.⁵ [symbol] Visvanathan, Karthik, and Yogesh B. Gianchandani “Microheaters based on ultrasonic actuation of piezoceramic elements.” Journal of Micromechanics and Microengineering 21.8 (2011): 085030.

Finally, the heating module 20 may generate heat via RF or microwave activation. In this version, the gel package would be placed within an RF or microwave-generating device. The casing [22] would need to be made from material that is compatible with RF or microwave energy generation. Furthermore, there would need to be safety features, such as overtemp protection and/or room for expansion of the gel as it heats within the compartment [18] in order to prevent the inadvertent explosion of a gel package during the heating process.

If we now turn to FIG. 6, we can more closely examine the features of the chemically activated heating module version 300. FIG. 6 is a perspective view of a preferred embodiment of a chemically activated version 16A of the gel packet of FIG. 4.

T The outer casing 22 will house a two-part gel compartment 18. As discussed previously, there will be a gel compartment 18 and a heating component compartment 20A. Part A of the gel 24 will be in the gel compartment 18. Part B of the gel 26 will be contained within the heating component compartment 20A. The two compartments 18, 20A will be separated by a separation portion 29 of the outer casing 22.

In its preferred form, the packet 18A will have a frangible seal portion 28 that makes up at least a part of the separation portion 29. This frangible seal portion 28 is designed so that the operator can cause a port to be created between the gel compartment 18 and heating component compartment 20A without breaking open the outer casing 22. Once the port between the two compartments 18, 20A is created (e.g. by some sort of hand manipulation of the package 18A by the operator), the two parts of the gel 24, 26 will come into contact with one another so that they will mix. The parts 24, 26 are designed to generate heat when they mix with each other.

There are several possible chemical compositions for warming gel. One possible composition utilizes an acid-base neutralization reaction. These reactions are typically highly exothermic when between a weak acid and strong base, or between a strong acid and weak base. Depending on the specific acids and bases used, the composition could be engineered to leave a neutral pH reaction—this ideally would stay in solution and be non-toxic (e.g. the resulting products of the reaction would be saltwater). This would require a gel, an acidic solution, and a basic solution to be stored in separate compartments prior to activation (each compartment would be separated from the others by inter-compartmental seals/walls). To activate heat generation, the seal between the acid and base would be broken, allowing the neutralization reaction to take place, producing a salt solution hotter than 40° C., which would then be mixed with a very thick gel by breaking a second frangible seal in the package containing the gel.

Another possible form of heating the gel utilizes an exothermic reaction via introduction of catalyst(s) whereby heat is produced through polymerizations. In a preferred form, the dissolution of an anhydrous salt into the gel is an effective method of warming. The two salts that have the highest enthalpy of solution are calcium chloride and magnesium chloride. On the assumption that the mass of the gel is 30 g and that its heat capacity is 4.18 J/(K·g), the required enthalpy to heat the gel from 21° C. to 40° C. is calculated using Equation 1 below:

$$Δ H = {c}_{p} \cdot m \cdot Δ T = 4.18 \frac{J}{K \cdot g} \cdot 30 g \cdot 19 K = 2383 J$$

Based upon the standard enthalpies of formation, the standard enthalpies of the solution for anhydrous calcium chloride and anhydrous magnesium chloride are −82.0 kJ/mol and −155.0 kJ/mol, respectively. Thus, using the molar masses of the two salts, the amount of salt needed to achieve a 19° C. temperature increase is 3.23 g of calcium chloride or 1.46 g of magnesium chloride. The difference in masses between salt types shows that the use of magnesium chloride would decrease the mass of salts required and, as discussed below, would likely decrease both the activation time and the introduction of air into the gel. The theoretical numbers for each salt are detailed in Table I below.

TABLE I
Theoretical Salt Requirements

| | Standard | | |
|---|---|---|---|
| | enthalpy of | Molar | |
| | solution | Mass | Required |
| Compound | (kJ/mol) | (g) | Mass (g) |
| Calcium Chloride | −82.0 | 111.0 | 3.23 |
| (anhydrous) | | | |
| Magnesium | −155.0 | 95.2 | 1.46 |
| Chloride | | | |
| (anhydrous) | | | |

Several additional viable formulations have been developed, with all containing similar ingredients. All of the formulations contain a cellulosic thickener, glycerin, deionized water, sodium benzoate, and an anhydrous salt. Two different thickeners were used including Natrosol 250 HHR (a hydroxyethylcellulose), and KELTROL CG-T (a xanthan gum). Both of the thickeners are used in a wide range of products, and are very pH stable. Because these thickener products are nonionic, their combination with salts will not affect their ability to thicken into a gel. Glycerin, which acts as a stabilizer and emulsifier, is also a common additive in many cosmetic formulations. It aids in keeping the hydroxyethylcellulose or xanthan gum stable in the gel. Sodium benzoate, which is a common preservative and is compatible with both Natrosol 250 HHR and xanthan gum.

Continuing with preferred form options, a two-part system for heating of clear gel by the addition of an anhydrous salt is described. This is a two-part formulation which consists of a base gel, which, when mixed with an anhydrous salt, heats up to about 40° C. (depending on ambient storage temperature). After activation, these gel formulations have viscosities at 37° C. that are similar to current products on the market, such as Aquasonic 100, Aquasonic Clear, and Clear Image Singles, which all range from 35000-41000 cP. Two variations of this formulation were prepared and tested. The first was a two-part formulation which consisted of a gel made with hydroxyethylcellulose (HEC, Natrosol 250 HHR), glycerin, and sodium benzoate. Hydroxyethylcellulose is a cellulosic thickener used in a wide range of industries and used in levels ranging from 0.1-3.0%. It is stable in the pH range of 2-12, and its ability to thicken into a gel is nonionic, therefore salts do not affect its viscosity. Glycerin is a common additive to many cosmetic formulations as a stabilizer and emulsifier that stabilizes the hydroxyethylcellulose within the gel. Sodium benzoate is a common preservative and is compatible with Natrosol 250 HHR. For a 30 g transmission gel, a base gel containing 96.10% deionized water, 2.62% hydroxyethylcellulose, 1.16% glycerin, and 0.12% sodium benzoate was made. Next, 4.05 g of anhydrous calcium chloride was added, raising the temperature to from 23.4° C. to 41.6° C. It took approximately 45 seconds to activate, and the final viscosity after activation at 37° C. was 40800 cP.

A second iteration of this two-part formulation utilized anhydrous magnesium chloride instead of anhydrous calcium chloride. The base gel for this formulation kept the ratio of the hydroxyethylcellulose to water in the base gel the same while keeping the percent of glycerin and sodium benzoate the same in the overall formula. For a 30 g transmission gel, a base gel containing 96.21% deionized water, 2.62% hydroxyethylcellulose, 1.16% glycerin, and 0.12% sodium benzoate was made according to the section below. Next, 1.8 g of anhydrous magnesium chloride was added, raising the temperature to from 22.9° C. to 41.6° C. It took approximately 15 seconds to activate, and the final viscosity after activation at 37° C. was 42800 cP. Because anhydrous magnesium chloride has a much higher standard enthalpy of solution, much less material was needed to achieve the same temperature increase as when anhydrous calcium chloride was substituted. Both the anhydrous calcium chloride and the anhydrous magnesium chloride required about 1.4-1.6 times the theoretical amount of salt (based off thermodynamic data found using Equation 1 and data in Table I), most likely due to heat loss through the package during the activation period, but also possibly due to unavoidable water uptake from exposure to atmosphere during testing. Handling the salts under inert gas during the manufacturing/packaging process might reduce the amount of salt required.

Another option is the formation of a gel by mixing a powder with a second component that is in liquid phase. This requires the use of a thickener that has a lower required temperature for activation. KELTROL CG-T is a xanthan gum specifically designed to produce a more transparent gel than most other xanthan gums. Both KELTROL CG-T and the anhydrous salts tested were in the form of a fine powder, so these were mixed to form a homogeneous powder. The water, glycerin, and sodium benzoate were mixed together and then added to the powdered components and then mixed continuously. The temperature immediately rose to 44.1° C. (this slightly higher temperature was chosen in order to help decrease hydration time of the thickener as well as compensate for heat loss through the packaging during the longer activation time). This option would decrease manufacturing costs considerably, but at the cost of a longer activation time (1.5-2 min). Additionally, the gel is not as transparent and is therefore visually distinct as compared to current gel products on the market.

A three-phase system for gel warming is described. This option used the same ingredient percentages as Formulations 1 and 2, but separates it into 3 phases in order to decrease the activation time. An amount of water (roughly 40% for each formulation) equal to that from Phase A would be kept as a separate phase (Phase C). Activation of this formula consists of mixing Part B and Part C together, and then be adding this mixture to Part A, and then mixing thoroughly. This decreased the activation time significantly because the dissolution of the salt into water was much faster than dissolution of the salt into a gel. However, the drawback was the final gel formed was not homogenous due to Phase A being too thick. Natrosol 250 HHR has a maximum usage of 3.0%, but when the water is not included in Phase A, Natrosol 250 HHR content increases to about 4.5%. At this concentration the hydroxyethylcellulose is not fully hydrated and the resulting gel is too thick and very clumpy. In order for the further addition of water to be effective, the Natrosol 250 HHR would still need to be fully hydrated, which requires time and a temperature of at least 55° C., thus when Phase CB were mixed in with this thicker form of Phase A, they remained separate and a homogeneous gel was not obtained.

Once the now-mixed gel has reached the desired temperature, the operator opens the casing 22, such as by cutting or tearing off the end of the packet 18A along a line 30 that denotes an end of the two compartments 18, 20A, as depicted here. The heated gel is applied directly to the patient's body, and then the used package 18A is discarded.

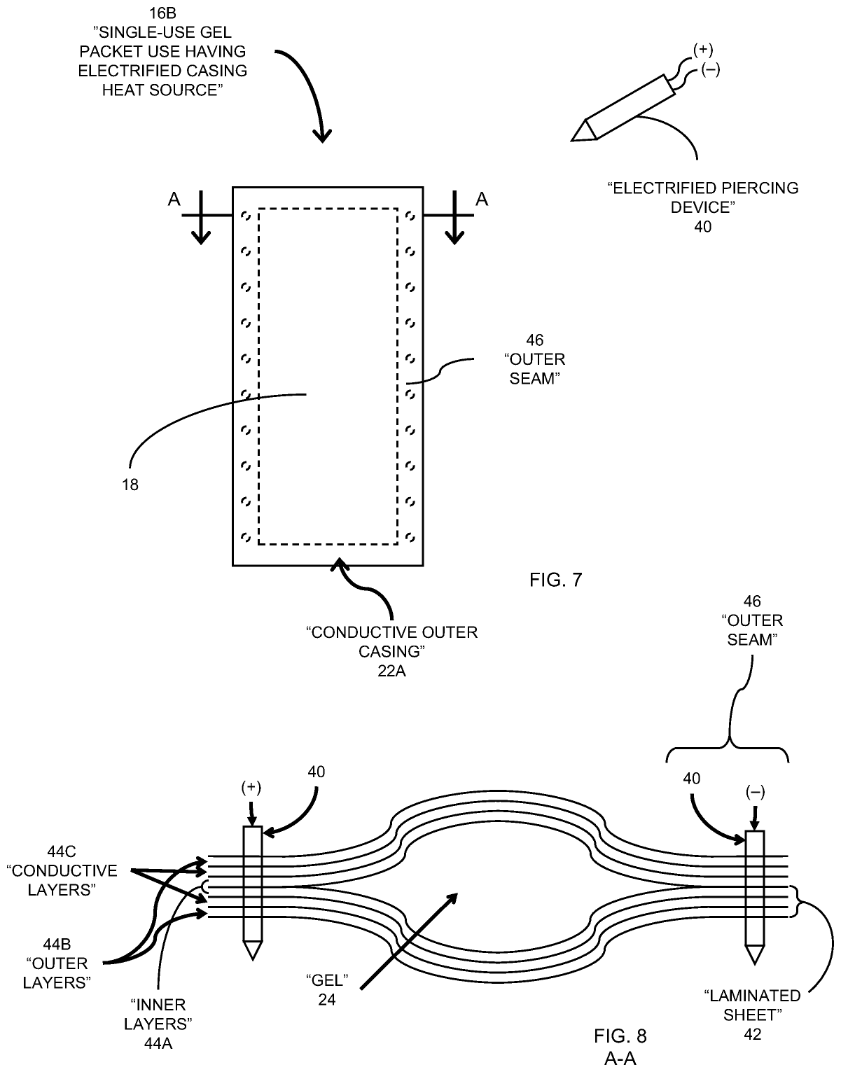

FIG. 7 is a front view of a preferred embodiment of a single-use gel packet having electrified casing heat source 16B. This packet 16B has an outer casing 22A that is configured to generate heat once electrical power (such as from electrified piercing device 40) is applied to the casing 22A. Gel compartment 18 is a sealed compartment containing the gel material that will be heated by the heat generated by the electrified outer casing 22A.

The electrified piercing device 40 could be bi-polar—having both positive and negative portions (separated by a non-conductor), or it could be two separate uni-polar devices 40 (as shown in FIG. 8). FIG. 8 is a partial cutaway end view of the packet 16B of FIG. 7. The casing 22A of the packet 16B is preferably formed from a pair of laminated sheets 42. Each laminated sheet 42 consists of an inner layer 44A, an outer layer 44C, and a conductive layer 44B sandwiched between the two. The inner layer 44A and outer layer 44C are both formed from pliable material that is sterile, non-electrically-conductive, and non-reactive to the gel material 24 within the compartment 18. The conductive layer 44B contains a plurality of conductive filaments (e.g. a mesh of electrical conductor filaments) that extend over the length and width of the casing 22A. When one (bi-polar) or a pair (uni-polar) piercing device(s) 40 is punched through the layers 44A, 44B, 44C of the outer casing 22A, electrical power energizes the filaments of the conductive layers 44C, and begins to generate heat. These piercing device(s) 40 are intended to pierce the outer seam 46 of the packet 16B without puncturing the gel compartment 18. This leaves the gel 24 in a sterile state until such time as the technician tears open the packet in order to dispense the gel material 24.

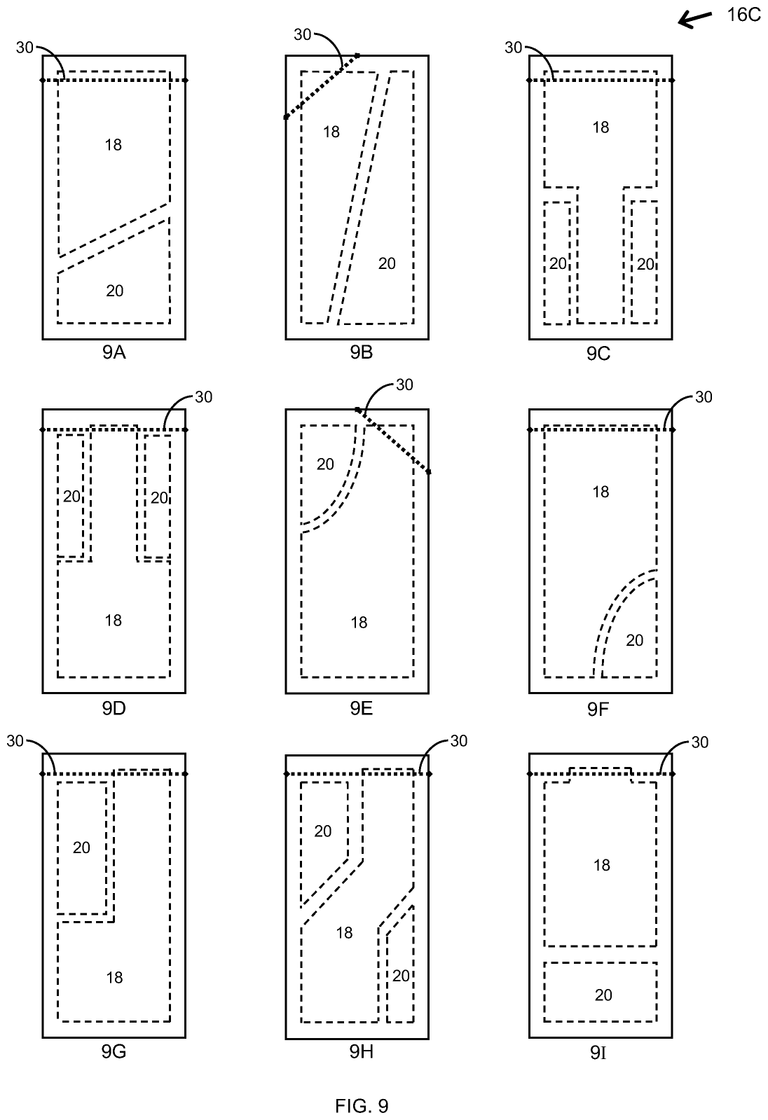

FIG. 9 are front views of several alternate versions of the single-use gel packet having an internal heat source. In each of these versions, the tear line 30 does not traverse the heating component compartment 20, but rather only traverses the gel compartment 18. Some or all of the barrier(s) between the gel compartments 18 and the heating component compartments 20 of these packets 16C is frangible. This means that by squeezing the packet 16C (or a portion of the packet 16B), the frangible barrier will be broken. This will allow the gel and the heating component to mix—thereby creating the chemical reaction that generates heat. Once the two parts are mixed sufficiently, and the desired heat has been generated, the technician can tear open the packet 16C at the tear line 30 to dispense the heated gel.

Those skilled in the art will appreciate that various adaptations and modifications of the just-described preferred embodiment can be configured without departing from the scope and spirit of the invention. Therefore, it is to be understood that, within the scope of the appended claims, the invention may be practiced other than as specifically described herein.

### APPENDIX I

“Fomites and Infection transmission” Infection Control Today magazine Nov. 7, 2006 In a systematic review of the literature, German researchers explored the ability of infectious organisms to survive on inanimate surfaces. They found that most gram positive bacteria, including VRE, MRSA and Streptococcus pyogenes can survive for months on dry surfaces.” “In the hospital environment, surfaces with which hands come in contact are often contaminated with Nosocomial pathogens, and may serve as vectors for cross-contamination.” (A fomite is any object that may be contaminated with infectious organisms and serve in their transmission.)
“Sustained Endemicity of Burkholderia Cepacia Complex in a Pediatric Institution, Associated with Contaminated Ultrasound Gel.”” Infection Control Hospital Epidemiology 2006, Apr. 27; 362-6 Conclusions: Contaminated ultrasound gel contributed to nosocomial infection due to B. cepacia complex in this institution over the course of 10 years.
“Sonographers and the Fight Against Nosocomial Infections: How Are We Doing?” JDMS 21: 7-11, January/February 2005: One in six (16.7%) gel bottle tips contaminated with bacteria/fungi.
Health Products and Food Branch of Canada Safety Alert October 2004: Risk of Serious Infection from Ultrasound and Medical Gels—Notice to Hospitals. In order to minimize the health risks associated with the use of ultrasound gels, Health Canada is making the following recommendations: Single Use Containers are recommended for non-sterile gel.
“An Outbreak of Pyodermas Among Neonates caused by Ultrasound Gel Contaminated with Methicillin-Susceptible Staphylococcus Aureus.” December 2000 Infection Control and Hospital Epidemiology Volume 21, No. 12. Conclusion: Inappropriate hygienic measures in connection with lubricants (gels) during routine ultrasound scanning may lead to nosocomial S aureus infections of the skin.
“An Investigation of the microbiological contamination of ultrasound equipment” Sykes A', Appleby M, Perry J, Gould K. British Journal of Infection Control August 2006 Vol 7 NO. 4. This study was undertaken to determine the extent of contamination of ultrasound equipment including probe, probe holder, keyboard and gel. The results revealed that 64.5% of the samples were contaminated with environmental organisms, 7.7% with potential pathogens and 27.8% were no growth.
“Iatrogenic urinary tract infection with Pseudomonas Cepacia after transrectal ultrasound guided needle biopsy of the prostate” Journal of Urology 1993, March; 149(3) “Environmental investigations revealed the ultrasound transmission gel as the source of the contamination.”
“Nosocomial Outbreak of Klebsiella pneumoniae Producing SHV-5 Extended-Spectrum-Lactimase, Originating from a Contaminated Ultrasonography Coupling Gel.”” Journal of Clinical Microbiology, May 1998 p 1357 -1360 “To summarize, this outbreak showed unique features, i.e., very early cross-contamination, lack of recognized risk factors of patients, transmission of the multiresistant strain by a soiled ultrasonography coupling gel, and intrapartum contamination of two neonates. It highlights the necessity of evaluation of nosocomial risks associated with procedures with good safety records such as ultrasonography.”
“Ultrasound Instruments as Possible Vectors of Staphylococcal Infection.”” Journal of Hospital Infection Control, September 1998, 40 (1) 73-7 “S. aureus was more resistant to the ultrasonic medium than Pseudomonas aeruginosa, also a significant cause of hospital-acquired infections.”
“Risk of Staphylococcus Aureus Transmission during Ultrasound Investigation,” Journal of Ultrasound in Medicine, November 1989 8 (11): 619 20. “An investigation of staphylococcal transmission between patients undergoing routine abdominal ultrasound procedures was carried out to assess the potential risks of cross-infection. Two of 19 patients were shown to have become colonized as a result of the procedure and some evidence is presented of possible interpatient cross-colonization. It is suggested that this is an underestimation of the potential of cross-infection and simple inexpensive infection control procedures are suggested”
“Burkholderia cepacia infections associated with intrinsically contaminated ultrasound gel: the role of microbial degradation of parabens.” Infection Control and Hospital Epidemiology. 2004 April; 25(4):291-6. CONCLUSIONS: Ultrasound gel is a potential source of infection.
“Physical methods of reducing the transmission of nosocomial infections via ultrasound and probe.” Clinical Radiology 1998 March; 53(3):212-4 “Nosocomial infections are posing an increasingly serious problem in the hospital setting. With the increasing use of ultrasound in medical diagnosis, there is the potential for transmission of nosocomial infections via the ultrasound transducer and coupling gel.”
APIC Archives established mechanisms of Gel Contamination including:
Contamination during dispensing practices—i.e., use of large containers purchased as a bulk supply and refilling smaller containers as needed for daily use.
Environmental contamination via nipple style tip.
Contamination at point of use—a patient or ultrasound probe could harbor pathogenic organisms. The application of gel involves squeezing the bottle to dispense gel. If the bottle is not withdrawn before pressure is released, the vacuum formed within the bottle could aspirate both gel and flora from the surface of the patient or the probe into the bottle.
   “Ultrasound Gel: a breeding ground for germs?” ADVANCE for Imaging and Radiation Therapy Professionals, Jun. 3, 2002
   An Epidemic, Toxin Gene—Variant Strain of Clostridium difficile, L. Clifford McDonald, M.D., George E. Killgore, Dr.P.H., Angela Thompson, M.M.Sc., Robert C. Owens, Jr., Pharm.D., Sophia V. Kazakova, M.D., M.P.H., Ph.D., Susan P. Sambol, M.T., Stuart Johnson, M.D., and Dale N. Gerding, M.D.
A previously uncommon strain of C. difficile with variations in toxin genes has become more resistant to fluoroquinolones and has emerged as a cause of geographically dispersed outbreaks of C. difficile-associated disease. Strict infection-control measures, including contact precautions, should be instituted for all patients with C. difficile-associated disease.
This article highlights the risks of using multiple use gel bottles where they are used on successive patients and Ultrasound Technicians touch the patient and the outside of the gel bottle with their gloved hands. The outside of the gel bottle becomes contaminated with microorganisms, such as C. difficile, and a vector for spreading infection.
   (Source Sonotech, Inc. “Journal Citation—Infection Control Risk with Bottles of Ultrasound Scanning Gel:”

What is claimed is:

1. A method for applying heated gelatinous material to the skin of a patient, comprising the steps of:
obtaining a container containing gelatinous material and a heating component, wherein said container comprises:
a pair of pliable walls bonded to one another around their periphery to form a packet, said packet defined by a top bonded seam and a bottom bonded seam;
a gel compartment formed within said packet, said gel compartment defined by upper and lower walls, with said upper wall defined by said top bonded seam;
a cut/tear line along said top bonded seam in relative spaced relation thereto;
a heating component compartment formed within said packet and wherein said cut/tear line does not pass over any portion of said heating component compartment; and
a frangible seal separating said gel compartment from said heating component compartment;
activating said heating component by squeezing said packet to breach said frangible seal and mixing said gelatinous material and said heating component whereby said mixed material and heating component produces heat;
waiting for a sufficient amount of time until said mixed gelatinous material and heating component is heated;
opening said container and applying said heated gelatinous material to the skin of the patient.

2. A self-heating gelatinous material packet assembly, comprising:
an outer casing comprising a pair of pliable sheets bonded to one another and having a peripheral seam forming an internal gel compartment, wherein each of said pliable sheets comprises an electrically-conductive layer; and
an electrified piercing device configured to apply electrical power to said electrically-conductive layers when said electrified piercing device pierces said outer casing.

3. The packet assembly of claim 2, wherein said outer casing comprises an outer layer external to said electrically-conductive layers, said outer layer is non-electrically conductive.

4. The packet assembly of claim 3, wherein said outer casing comprises an inner layer internal to said electrically-conductive layers, said inner layer is non-electrically conductive.

5. The packet assembly of claim 2, wherein said electrified piercing device comprises a tip suitable for piercing said outer casing and an exterior surface in electrical contact with positive and negative electrical terminals.

6. The packet assembly of claim 2, comprising two said electrified piercing devices, with each said piercing device comprising a tip suitable for piercing said outer casing and one said piercing device having an exterior surface in electrical contact with a positive electrical terminal and one said piercing device having an exterior surface in electrical contact with a negative electrical terminal.

7. The packet assembly of claim 2, wherein said electrical conductive layer comprises a series of small conductive filaments extending over between at least two of sides of said peripheral seam.

8. An ultrasonic gel packet, comprising:
a pair of pliable walls bonded to one another around their periphery to form a packet, said packet defined by a top bonded seam and a bottom bonded seam;
a gel compartment formed within said packet, said gel compartment defined by upper and lower walls, with said upper wall defined by said top bonded seam;
a cut/tear line along said top bonded seam in relative spaced relation thereto;
a heating component compartment formed within said packet and wherein said cut/tear line does not pass over any portion of said heating component compartment; and
a frangible seal separating said gel compartment from said heating component compartment.

9. The packet of claim 8, comprising two or more said heating component compartments adjacent to said gel compartment and separated therefrom by one or more said frangible seals.

10. The packet of claim 9, wherein said gel compartment defines a “T”-shape having a top portion that is wider than a lower portion extending downwardly therefrom.

11. The packet of claim 10, comprising a pair of said heating component compartments in spaced relation to, and on opposing sides of said lower portion of said gel compartment.

12. The packet of claim 9, wherein said gel compartment defines an “inverted T”-shape having a lower portion that is wider than a top portion extending upwardly therefrom.

13. The packet of claim 12, comprising a pair of said heating component compartments in spaced relation to, and on opposing sides of said top portion of said gel compartment.

14. The packet of claim 9, wherein said packet is further defined by opposing first and second bonded side seam, and wherein said gel compartment defines a “three-bar polygon”-shape having a lower portion extending upwardly generally parallel to said first side seam, a central slanted portion extending at a slant from said lower portion to an upper portion extending upwardly generally parallel to said second side seam.

15. The packet of claim 14, comprising a pair of said heating component compartments, one said heating component compartment disposed between said upper portion of said gel compartment and said first side seam, and the other said heating component compartment disposed between said lower portion of said gel compartment and said second second side seam.

16. The packet of claim 8, wherein said gel compartment comprises a generally rectangular shape, wherein said top bonded seam defines a top wall of said gel compartment, and said top wall comprises a portion of said top wall that is bisected by said cut/tear line and at least one portion of said top wall that is not bisected by said cut/tear line.
